(19) United States
(12) Patent Application Publication
(10) Pub. No.: US 2025/0002975 A1
(43) Pub. Date: Jan. 2, 2025

(54) DEVICE FOR DETECTING FLUORESCENCE WITH NANOPHOTODETECTORS

(71) Applicants: Commissariat à l'Energie Atomique et aux Energies Alternatives, Paris (FR); CENTRE NATIONAL DE LA RECHERCHE SCIENTIFIQUE, Paris (FR); UNIVERSITE GRENOBLE ALPES, Saint-Martin-D’heres (FR)

(72) Inventors: Olivier CONSTANTIN, Grenoble cedex 09 (FR); Moira HOCEVAR, Grenoble (FR); Pascal MAILLEY, Grenoble cedex 09 (FR); Eva MONROY, Grenoble cedex 09 (FR)

(73) Assignees: Commissariat à l'Energie Atomique et aux Energies Alternatives, Paris (FR); CENTRE NATIONAL DE LA RECHERCHE SCIENTIFIQUE, Paris (FR); UNIVERSITE GRENOBLE ALPES, Saint-Martin-D’heres (FR)

(21) Appl. No.: 18/754,303

(22) Filed: Jun. 26, 2024

(30) Foreign Application Priority Data
Jun. 27, 2023 (FR) 2306747

(51) Int. Cl. C12Q 1/6816; G01N 21/64; G01N 33/58

(52) U.S. Cl. CPC C12Q 1/6816; G01N 21/6428; G01N 21/6486; G01N 33/582; G01N 2021/6439

(57) ABSTRACT

The invention relates to a device for identifying a fluorescent label. The detection device comprises nanowires, each nanowire acting as a transducer of fluorescence light into an electrical signal. One targeted application is sequencing of a nucleic acid.

### TECHNICAL FIELD

The technical field of the invention is detection of biomolecules via optical transduction performed with nanowires.

### PRIOR ART

Devices allowing biomolecules to be detected may be based on optical detection. The biomolecules to be detected are labelled beforehand using a luminescent label, such as a fluorophore or quantum-dot label. Detection is achieved via a photodetector array such as a CCD or CMOS imager or an array of avalanche photodetectors (APDs).

A widespread application of fluorescent labelling is sequencing, in particular so-called SBS sequencing (SBS standing for Sequencing By Synthesis). According to this method, a sequence of single-stranded nucleotides, placed downstream of a primer, is amplified to form a cluster. During sequencing, a complementary sequence is gradually synthesized, by successively adding nucleic bases complementary to those forming the studied sequence. The complementary bases are added one by one, in respective cycles. After each addition of a complementary base, an image of the cluster is taken. All or some of the complementary bases are labelled with a fluorescent label. Observing the fluorescence in each cycle allows the complementary base added in said cycle to be identified. Thus, by taking a succession of fluorescence images, it is possible to decode the examined nucleotide sequence.

The fluorescence is detected by a photodetector, usually a photodetector array. However, the detector arrays of such photodetectors generally have a spatial pitch greater than 1 μm. By spatial pitch, what is meant is the distance between two adjacent pixels. Such a spatial pitch is considered to be too large to obtain a device allowing a large number of detections in parallel. These devices also have a limited sensitivity: it is necessary to provide sufficient amplification to obtain a sufficient number of identical sequences at the studied cluster, to obtain an exploitable signal.

Certain biosensors are based on detection of charges carried by the target biomolecules to be detected. It is a question for example of field-effect transistors. This type of device allows rapid detection, with a good sensitivity. In addition, CMOS technology (CMOS standing for Complementary Metal-Oxide-Semiconductor) allows devices with a large number of sensors to be manufactured. It is thus possible to carry out a large number of analyzes in parallel. However, this type of sensor may be sensitive to environmental parameters affecting the sample, for example pH or temperature or interactions with non-targeted biomolecules or ions inducing detection errors. Use of this type of biosensor in DNA sequencing applications (DNA standing for DeoxyriboNucleic Acid) requires a certain redundancy in the measurements, so as to increase their robustness. DNA sequencers based on nanopores have the same drawbacks.

Nanowire-based photonic sensors have been developed for the purpose of detecting DNA fragments. This is for example the case in the publication Sing, et al., “Silicon nanowire optical rectangular waveguide biosensor for DNA hybridization”, IEEE Photonics Technology Letters, 2018, 30 (12) 1123-1126. In this publication, DNA hybridization is detected by detecting a variation in refractive index during hybridization.

The publication Irrera, et al., “New generation of ultrasensitive label-free optical Si nanowire-based biosensors”, ACS Photonics 2018, 5 (2) 471-479 describes a nanowire-based biosensor used to detect CRP (C-reactive protein) in human serum.

Applications related to DNA sequencing require fluorescence coding, to differentiate the added bases. In applications employing a sensor, coding is achieved either through the emission wavelength, or through a light intensity in the image acquired by the photodetector.

The inventors have designed a device for analyzing a sample that is compact and sensitive, allowing advantage to be taken of the fluorescence-detecting capabilities of nanowires. The device may allow simultaneous or sequential analysis of various biomolecules. The device makes it possible to benefit from the detection sensitivity and compactness of nanowires, while allowing differentiated detection of nucleic bases labelled with fluorescent labels.

### DISCLOSURE OF THE INVENTION

A first subject of the invention is a device for identifying a fluorescent label, the fluorescent label being configured to emit fluorescence light in a fluorescence spectral band, the device comprising:
a substrate, comprising at least one first electrode;
a multilayer structure, comprising at least one second electrode;
nanowires, extending between the first electrode and the second electrode, parallel to a transverse axis;
an encapsulation layer extending around the nanowires, between the substrate and the multilayer structure, the encapsulation layer being formed from an insulating material;
the multilayer structure comprising:
   a conductive layer, forming each second electrode;
an electrically insulating interface layer covering each second electrode, each second electrode being interposed between the interface layer and one nanowire, the interface layer being bounded by a functionalization surface, the interface layer being configured to be placed between a sample, comprising the fluorescent label, and the second electrode, such that the functionalization surface forms an interface between the device and the sample;
the multilayer structure being such that the second electrode and the interface layer are transparent in a detection spectral band containing the fluorescence spectral band;
   the device being such that:
each nanowire comprises a homojunction, or a heterojunction, or a Schottky junction between the first electrode and the second electrode;
the first electrode and the second electrode are configured to be connected to a detection circuit;
in such a way that each nanowire forms a nanophotodetector of the fluorescence light when the fluorescent label is bound to the functionalization surface, the light detected by each nanowire inducing an electrical detection signal in the detection circuit;
   the device comprising a processing unit, programmed to:
acquire the detection signal during a detection time period;
determine a characteristic of the detection signal during the detection time period;
identify the fluorescent label depending on the characteristic.

According to one possibility, the device comprises a light source configured to emit excitation light in the excitation spectral band of the fluorescent label.

According to one possibility:
the light source is configured to emit excitation light during an excitation time period;
the detection time period is subsequent to the excitation time period.
   According to one possibility:
following the excitation time period, the intensity of the fluorescence light grows then decays;
the characteristic of the detection signal is representative of the decay of the intensity of fluorescence light.
   According to one possibility, the functionalization surface is configured to capture a strand forming a chain of oligonucleotides.

According to one possibility, each nanowire comprises:
a p-n homojunction;
or a heterojunction;
or a p-metal or n-metal Schottky junction.

According to one possibility, at least one nanowire comprises a first portion and a second portion, which are separated by the junction, the first portion being formed from an n-doped semiconductor, the second portion being formed from a p-doped semiconductor, the junction forming a homojunction or a heterojunction.

According to one possibility, at least one nanowire comprises a first portion and a second portion, which are separated by the junction, the first portion being formed from a semiconductor, the second portion, adjacent to an electrode, being formed from a metal, the junction forming a Schottky junction.

According to one possibility, the first portion and the second portion extend, along the transverse axis, on either side of the junction.

According to one possibility, the second portion encircles the first portion, around the transverse axis.

According to one possibility, the functionalization surface is segmented into various capture sites, each capture site being configured to capture one strand forming a chain of oligonucleotides.

According to one possibility:
the interface layer comprises two sub-layers stacked on top of each other, forming a lower sub-layer and an upper sub-layer, the lower sub-layer being interposed between the conductive layer and the upper sub-layer;
the upper sub-layer comprises wells that open into the lower sub-layer, each well being placed facing one nanowire, each well forming part of the functionalization surface;
the functionalization surface is segmented at the level of each well, so that each well forms one capture site.

According to one possibility, the device comprises a plurality of nanowires, extending between the same first electrode and the same second electrode, the nanowires forming a nanowire cluster.

According to one possibility, the device comprises a plurality of nanowire clusters spaced apart from one another, such that one nanowire of a cluster is closer to another nanowire of said cluster than to another nanowire of another cluster, the nanowires of a given cluster extending between the same first electrode and the same second electrode.

According to one possibility, the device comprises a plurality of nanowires, the device being such that:
a plurality of first electrodes are formed on the substrate, and a plurality of second electrodes are formed on the multilayer structure, each nanowire extending between a first electrode and a second electrode;
each first electrode is connected to a first addressing unit, configured to select at least one first electrode;
each second electrode is connected to a second addressing unit, configured to select at least one second electrode;
such that the detection circuit detects a detection current induced by each nanowire extending between the selected first electrode and the selected second electrode.

A second subject of the invention is a method for identifying a fluorescent label using a device according to the first subject of the invention, the fluorescent label being capable of emitting fluorescence light, in the detection spectral band, when it is illuminated by excitation light, the functionalization surface being configured to capture a strand of nucleic acid, the method comprising:
a) placing a sample, comprising nucleic acids, in contact with the functionalization surface;
b) capturing at least one strand of nucleic acid on the functionalization surface;
c) adding nucleic bases to the sample, at least two different nucleic bases being labelled with two different fluorescent labels, the sample comprising active principles configured to allow hybridization of a nucleic base to the strand of nucleic acid captured on the functionalization surface;
d) exposing the functionalization surface to excitation light, in an excitation spectral band of at least one fluorescent label;
e) following step d), detecting a detection signal across the terminals of the detection circuit, during a detection time period;
f) depending on the detection signal detected in step e), identifying the fluorescent label;
g) reiterating steps c) to f) so as to gradually hybridize nucleic bases along the strand of nucleic acid.

Step b) may comprise amplification of each captured strand of nucleic acid.

According to one possibility, steps b) to g) are carried out at various capture sites distributed over the functionalization surface.

According to one possibility, step f) comprises:
determining a characteristic of the detection signal during the detection time period;
identifying the fluorescent label depending on the characteristic.

According to one possibility:
the fluorescent label is chosen from a plurality of candidate fluorescent labels;
step f) comprises selecting the fluorescent label from the candidate fluorescent labels depending on the characteristic of the detection signal.

According to one possibility, step f) comprises estimating a time derivative of the detection signal.

According to one possibility, step f) comprises detecting an intensity level or integrating the detection signal during at least one predetermined time period. According to one possibility:
prior to step a), each nucleotide sequence is bound to a known calibration sequence;
steps c) to f) are implemented so as to hybridize the bases of the calibration sequence, steps c) to f) forming a calibration phase;
the detection signal obtained in each step e) of the calibration phase is used to calibrate a response of the device to the bases of the calibration sequence.

According to one possibility:
each candidate fluorescent label emits fluorescence light with a fluorescence intensity that grows then decays;
the decay of the fluorescence intensity of each fluorescent label is characterized by a decay constant;
the decay constants of two different fluorescent labels are different.

According to one possibility, following step g), the method comprises a step h) of identifying the hybridized nucleic acid base.

According to one possibility:
following step h), the fluorescent label is cleaved and the sample is rinsed;
after rinsing, steps c) and h) are reiterated, so as to identify a nucleotide sequence forming the captured strand of nucleic acid.

The invention will be better understood on reading the description of the examples of embodiment presented, in the remainder of the description, with reference to the figures listed below.

### FIGURES

FIGS. 1A and 1B show the main components of a device according to the invention.

FIG. 1C shows a spatial distribution of capture sites.

FIG. 1D shows one example of a device according to the invention, in which the electrodes are distributed in a matrix-array arrangement.

FIG. 1E shows one example of sequencing of three different sequences, on three capture sites, respectively.

FIG. 2A shows temporal distributions of emission of fluorescence light of various fluorescent labels.

FIGS. 2B and 2C show examples of characterization of the temporal distributions of emission of fluorescence light, the characterization being carried out during a detection time period.

FIG. 2D schematically shows various detection time periods during the decay of the emission of fluorescence light in one instance.

FIG. 3A illustrates a calibration sequence.

FIG. 3B shows various intensity levels detected during a calibration phase.

FIG. 3C schematically shows successive characterization signals, respectively obtained during sequencing of a TTCG sequence, with cleavage of the fluorescent labels in each cycle.

FIG. 3D schematically shows successive characterization signals, respectively obtained during sequencing of a TTCG sequence, without cleavage of the fluorescent labels in each cycle.

FIGS. 4A to 4E schematically show steps of manufacture of nanowires according to a so-called bottom-up process.

FIG. 5 shows various possible arrangements of the nanowires.

FIGS. 6A and 6B show two different nanowire structures.

FIG. 7 shows various steps of implementation of the method.

### DISCLOSURE OF PARTICULAR EMBODIMENTS

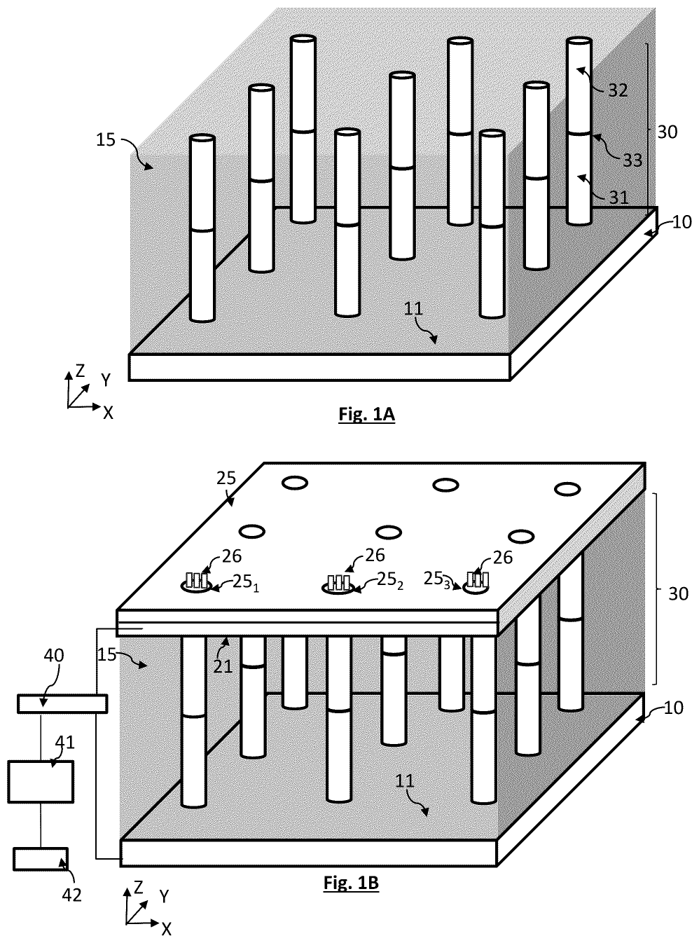

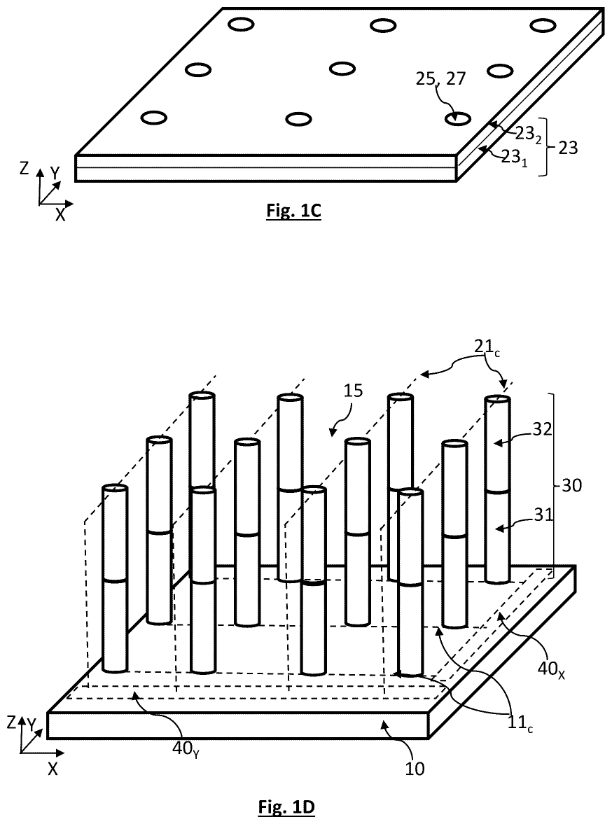

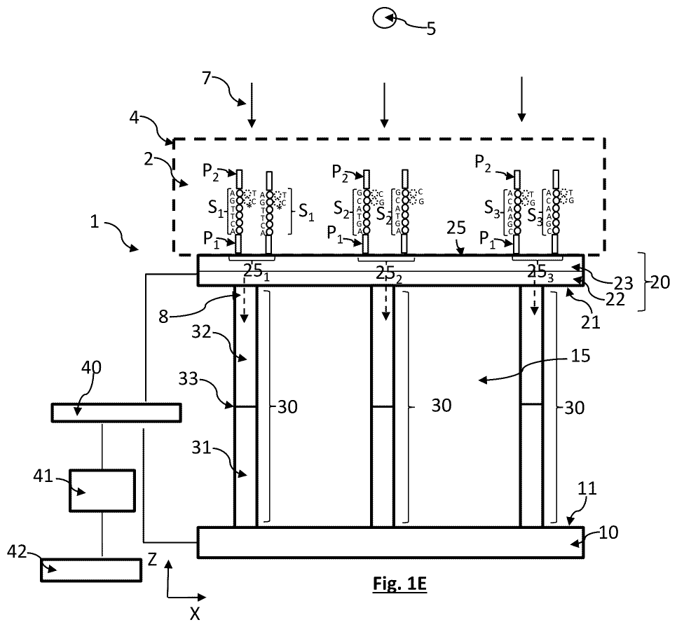

FIGS. 1A to 1E show a first example of an analysis device 1 allowing the invention to be implemented. The analysis device 1 is configured to be placed in contact with a sample 2. The sample for example comprises a liquid medium liable to contain strands of nucleic acids that it is desired to sequence. Each strand results from fragmentation of genetic material, forming a library of strands. Each strand may comprise an adapter at 3′ end and at the 5′ end. In a known manner, the adapter may comprise a primer binding site and optionally an index containing a code identifying the sample. One of the adapters may comprise a sequence referred to as the calibration sequence, which will be described below.

The device comprises a substrate 10, forming or comprising at least one first electrode 11_{c}. In the example shown in FIG. 1A, the substrate is a crystalline silicon substrate, for example an Si substrate of (111) crystal orientation. The substrate 10 is bounded by a surface, called the first surface 11, comprising a first electrode 11_{c}. In the example of FIG. 1A, the first surface 11 is formed from Si containing conductive regions. According to another possibility, the substrate 10 undergoes deposition of a conductive layer, for example of graphene, which forms all or part of the first surface 11.

Nanowires 30 are formed on the substrate 10, and more precisely from the first surface 11. The first surface 11 lies in a plane P_{XY}. The plane P_{XY} is defined by a longitudinal axis X and a lateral axis Y. The axes X and Y are secant, and preferably perpendicular to each other. The nanowires extend parallel to a transverse axis Z secant to the plane P_{XY}. In the embodiments described below, the transverse axis Z is perpendicular to the plane P_{XY}. The first surface 11 is conductive at least at the intersection with each nanowire 30. The entirety of the first surface 11 may be conductive.

According to other configurations, the nanowires may be inclined and not perpendicular to the plane P_{XY}. For example, if the crystal orientation of the material forming the substrate 10 is (001), the nanowires may grow in a (111) direction, and therefore slanted with respect to the plane P_{XY}.

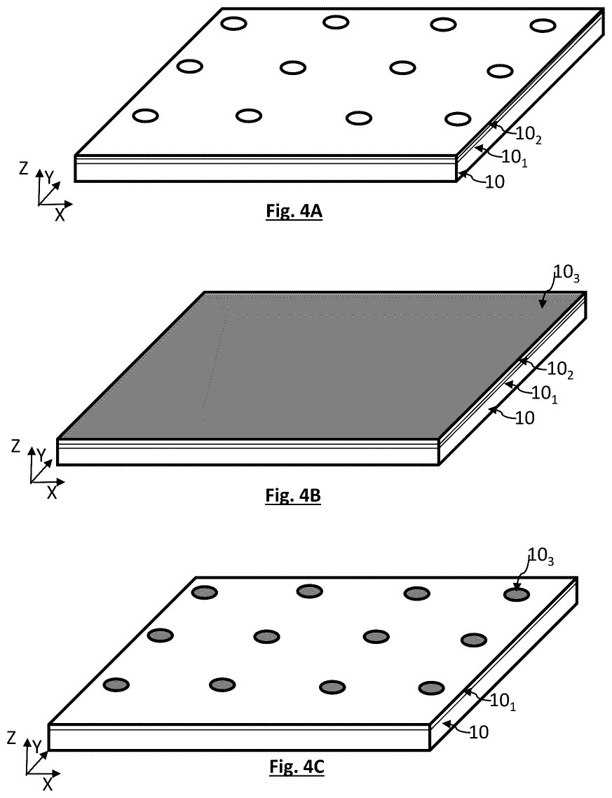

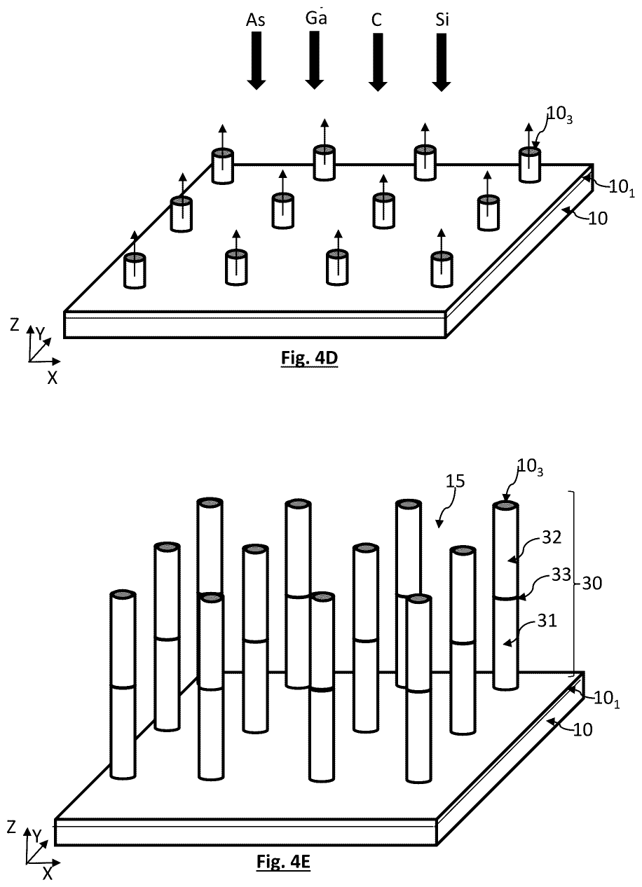

The nanowires 30 preferably have a diameter between 1 nm and 500 nm and a height, along the transverse axis Z, between 300 and 1000 nm, or even 10000 nm. The nanowires 30 may be synthesized directly on the substrate 10, as described with reference to FIGS. 4A to 4E. The nanowires may be formed on another substrate, then transferred to the substrate 10. The transfer may be carried out as described in the publication Valente, et al., “Light-Emitting GaAs Nanowires on a Flexible Substrate”, Nano Lett 2018, 18 (7) 4206-4213.

The nanowires 30 extend, from the first surface 11, to a second surface 21 bounding a multilayer structure 20. Just like the first surface 11, the second surface 21 is conductive at least at the intersection with each nanowire 30. In the example shown in FIG. 1B, the second surface 21 is formed from a layer 22 of a conductive material that is transparent in a detection spectral band described below. It may for example be a question of indium tin oxide (ITO).

Each nanowire is formed from one or more semiconductors, and optionally from a metal. Each nanowire comprises a junction 33. In the example shown, the junction 33 is a homojunction: each nanowire comprises a first portion 31, adjacent to the first surface 11, and a second portion 32, adjacent to the second surface 21. The first and second portions are formed from the same semiconductor, but have different respective dopings: thus, the first portion 31 and the second portion 32 are formed from the same semiconductor doped n and p or p and n, respectively. In the example shown, the first portion 31 is formed from p-doped gallium arsenide (GaAs) and the second portion is formed from n-doped GaAs. The interface between the two portions forms the p-n junction 33.

Alternatively, the junction 33 may be formed at a metal placed on or in contact with the surface 21, so as to establish a semiconductor-metal Schottky junction.

Each nanowire comprises at least one semiconductor chosen from the materials of columns Ill and V, usually designated by the term III-V materials, GaAs for example. It may preferably be a question of a material from column III and of arsenic, indium arsenide (InAs) for example. In the example shown, the nanowires 30 are formed from GaAs. Other semiconductors may be envisioned, for example, and non-limitingly, Si, InGaAs, AlGaAs, InGaP, InGaN, GaN, ZnSe, ZnS, ZnO, ZnCdO, ZnTe, CdSe, Ge, GeSn.

Between the first surface 11 and the second surface 21, the nanowires 30 are embedded in an encapsulation layer 15 formed from an insulating material. The encapsulation layer 15 may be formed from a material such as polymethyl methacrylate (PMMA) or benzocyclobutene (BCB), or such as a spin-on glass (SoG), spin-on glasses mainly consisting of silicon oxides and other chemical additives endowing them with specific properties such as adhesion and thermal stability. The encapsulation layer 15 may be deposited by spin coating.

The encapsulation layer 15 is preferably formed following growth of the nanowires, prior to deposition of a second conductive layer 22, bounded by the second surface 21, and intended to form second electrodes 21_{c}. The second electrodes 21_{c} are formed, on the second surface 21, from the conductive layer 22. The conductive layer 22 may be structured so that, on the second surface 21, a plurality of second electrodes 21_{c} are electrically insulated from one another. Thus, the conductive layer may comprise apertures or insulating materials delineating the electrodes 21_{c}. This allows differentiated detection at each nanowire.

Apart from the conductive layer 22, the multilayer structure 20 comprises an interface layer 23, adjacent to the conductive layer 22. The interface layer 23 is transparent in the detection spectral band. The interface layer 23 may for example be formed from a layer of a polymer, polymethyl methacrylate (PMMA) for example. The interface layer 23 is electrically insulating, in particular at the interface with the conductive layer 22. The interface layer 23 is preferably planar.

The interface layer 23 is intended to form an interface between the conductive layer 22, forming the electrodes 21_{c}, and the sample 2. Thus, the interface layer 23 lies between the conductive layer 22 and the sample 2. An important aspect of the device is that the sample does not make direct contact with the nanowires. It is isolated from the latter by the interface layer 23.

The interface layer 23 is for example a thin layer formed from SiO₂ or PMMA. The surface of the interface layer 23 intended to make contact with the sample is a surface, called the functionalization surface 25, that is functionalized by biological capture species 26. It is important for the interface layer 23 to be formed from a material having as low an autofluorescence as possible in the detection spectral band.

The functionalization surface 25, which forms an interface between the multilayer structure 20 and the sample 2 to be analyzed, is intended to be functionalized by capture species 26. By capture species, what is meant is a species configured to capture a biomolecule of interest, and more precisely a nucleotide sequence to be analyzed.

According to one possibility, the capture species is formed from oligonucleotides configured to graft to the nucleic acid to be analyzed. It may in particular comprise a chain of nucleotide bases complementary to all or some of the adapters bound to the strands to be characterized. The capture species 26 and each strand of oligonucleotides to be analyzed, or more precisely an adapter of each strand of oligonucleotides to be analyzed, then bind through hybridization. The interface layer 23 may be nanostructured, to form nanowells 27 at the interface between the multilayer structure 20 and the sample to be analyzed. The diameter or largest diagonal of the nanowells may be between 70 nm and 700 nm. The nanowells 27 may for example be arranged in a matrix array, or, more generally, in a predetermined pattern. The functionalization surface is then functionalized in each nanowell 27, the spaces between each well not being functionalized. The nanowells may be formed by thinning the interface layer 23 locally.

According to one possibility, shown in FIG. 1C, the interface layer 23 comprises two superposed sub-layers 231 and 232. The interface layer comprises a lower sub-layer 231 interposed between the conductive layer 22 and an upper sub-layer 232. The nanowells are formed by thinning the upper sub-layer 232 locally, so that the nanowells open into the lower sub-layer 231. The upper sub-layer 232 may be formed from a non-functionalizable material, for example an anti-biofouling material, a hydrophobic material for example. Such structuring allows the functionalization surface 25 to be functionalized solely in the nanowells 27, on the lower sub-layer 231. Each nanowell 27 thus forms a site for capturing a DNA strand.

More generally, the functionalization surface 25 may be functionalized in a predetermined functionalization pattern. Outside of the functionalization pattern, the functionalization surface is not functionalized

Functionalization may be achieved via a treatment of the functionalization surface 25, for example a plasma/oxygen surface treatment. When the interface layer 23 is formed from PMMA, a plasma/oxygen treatment makes it possible to form carboxyl functions. The capture species 26 may be grafted onto the functionalization surface by covalent bonding. To this end, the capture species comprise a function, for example an amine function, so as to form a covalent bond with the functions of the functionalization surface 25. It may for example be a question of a bond obtained by grafting a thiol function, of the capture species, to an amine function present on the functionalization surface.

According to one possibility, each sequence to be analyzed comprises a thiol function, for example at 3′ end, so as to form a covalent bond with the functionalization surface. In this case, the functionalization surface acts as capture species.

As shown in FIGS. 1B and 1E, the device comprises a detection circuit 40, a first terminal of which is connected to a first electrode 11_{c}, on the substrate 10, and a second terminal of which is connected to a second electrode 21_{c}, on the multilayer structure 20. The detection circuit 40 makes it possible to measure the potential difference, or an electric current, between the first electrode 11_{c} and the second electrode 21_{c}.

As described with reference to FIGS. 1A to 1C, each nanowire extends between a first end, on a first surface 11 of the substrate 10, and a second end, on a second surface 21 of the multilayer structure 20. The first surface 11 and the second surface 21 are conductive at least at each intersection with a nanowire. Thus, at each intersection with a nanowire, the first surface comprises a first electrode 11_{c} and the second surface comprises a second electrode 21_{c}. In the example shown in FIGS. 1A to 1B, the first surface 11 and the second surface 21 are formed from a conductive material. They are conductive over their entire area.

The first and second surfaces may be structured, and comprise various electrodes 11_{c}, 21_{c} that are insulated from one another. In FIG. 1D, each electrode has been represented by a dashed line. On the first surface 11, each first electrode 11_{c} describes one row, parallel to the longitudinal axis X. On the second surface 21, each second electrode 21_{c} describes one column, parallel to the lateral axis Y. Each nanowire is functional when the electrodes 11_{c}, 21_{c}, between which it extends, are biased. Structuring the electrodes in rows/columns makes it possible to select the functional nanowires, the latter extending between two biased electrodes: in this arrangement, the nanowires connected to a biased row and to a biased column are functional. By functional nanowire, what is meant is a nanowire biased to detect fluorescence photons.

A plurality of rows and/or a plurality of columns may be biased simultaneously or successively. The detection circuit 40 comprises:
an addressing unit 40_{X}, intended to bias all or some of the first electrodes 11_{c}, parallel to the axis X,
and an addressing unit 40_{Y} intended to bias all or some of the second electrodes 21_{c} parallel to the axis Y.

The functionalized portions of the functionalization surface form capture sites for the nucleotide sequences to be characterized.

As indicated above, each biological capture species 26 is configured to capture a biological species of interest, in particular a nucleotide sequence to be analyzed.

Preferably, after a nucleotide sequence has been captured at a capture site, said sequence is amplified, so as to obtain, at the same capture site, a plurality of replicas that are identical, excluding amplification errors, to the captured nucleotide sequence.

The amplification of each captured sequence may be bridge amplification, usually implemented in devices sold by the company Illumina. When bridge amplification is used, the functionalization surface bears capture species configured to graft to the adapters of the captured sequences.

FIG. 1E shows three sequences S₁, S₂ and S₃ captured, then amplified, on three capture sites 25₁, 25₂ and 25₃, respectively. Each nucleotide sequence to be characterized is for example obtained using a method for preparing a library of short DNA fragments, the length of which is typically 300 bases or 150 bases.

Generally, sequencing comprises a plurality of cycles, in which complementary bases are gradually hybridized along each sequence to be characterized. In a known manner, in each cycle, the sequences to be characterized are immersed in a reaction medium containing bases labelled with a fluorescent label and a hybridase. Hybridization takes place gradually along each strand, base by base, in a predetermined direction, for example from the free end to the end bound to the capture species.

According to one possibility, the fluorescent labels are coupled to a terminator, this preventing hybridization of two consecutive bases in the same cycle. The reaction medium then contains a mixture of bases, each base of a given type being labelled with the same fluorescent label. Two bases of different types are labelled with different fluorescent labels, respectively. Following each hybridization, the fluorescent label is detected after rinsing, this allowing the base hybridized in the cycle to be identified. Following detection, at least the terminator undergoes cleavage. The fluorescent label may also undergo cleavage. The medium is then rinsed and another cycle is initiated. The cycles are repeated until the sequences grafted at the various capture sites have been completely sequenced. Among the four different bases employed, one base may not be labelled.

According to another possibility, in each cycle, the sequences are successively immersed in baths, each bath containing a single type of base. Each base of a given type is labelled with the same fluorescent label. According to this possibility, it is possible, but not essential, for two bases of different types to be labelled with different labels, respectively. Specifically, each type of base is identified by each bath. Between each bath, a rinse is carried out, and fluorescence is detected at each capture site. According to this possibility, the fluorescent labels do not necessarily comprise terminators. Thus, a plurality of identical successive bases may be hybridized with complementary bases of the reaction medium. This results in a fluorescence signal the intensity of which increases as a function of the number of successive bases hybridized.

Following detection of a fluorescence signal, the fluorescent labels labelling each hybridized base in the cycle may be cleaved. As described below, cleavage is not essential.

Thus, at each capture site, each cycle comprises:
adding a reaction medium containing bases and a hybridase (polymerase):
either in the form of a mixture of bases of various types, in which case the fluorescent labels comprise a terminator;
or in the form of successive baths, each bath containing bases of the same type, in which case the fluorescent labels need not comprise a terminator;
rinsing;
detecting fluorescence (or absence of fluorescence) with the nanowires;
following detection of fluorescence, optionally cleaving the terminators if present and optionally the fluorescent labels, followed by rinsing.

Between each cycle, the reaction medium is washed and renewed. Each cluster is excited by a light source 5, in an excitation band of the fluorescent label or of each fluorescent label. The light source is configured to emit excitation light for a short time, of the order of one ns. It may for example be a question of a laser light source. This makes it possible to generate, at the level of each cluster, a fluorescence signal, which depends on the added base (A, C, T or G in the case of a DNA strand).

An important aspect of the invention, described below, consists in translating the fluorescence signal into an electrical signal dependent on the fluorescent label, this making it possible to obtain, in a cycle, an electrical signal dependent on the base added in the cycle.

This approach is substantially different from the sequencing approach based on formation of an image of the sample, each base being identified by a colour code and/or by an intensity level.

The invention takes advantage of the ability of a nanowire to detect an optical signal, in a predefined spectral band, and to convert the optical signal into an electrical detection signal. Thus, the device 1 is based on optical detection of hybridization of a base to a sequence to be analyzed, inducing an electrical response from the device. FIG. 1E shows a cross-sectional view of the device, after sequence amplification. It is assumed that, at each capture site, a single sequence has been captured (sequence S₁ at site 25₁, sequence S₂ at capture site 25₂, etc.). Each captured sequence was then bridge amplified. After amplification, identical sequences extend from each capture site, which sequences are formed at their 3′ end from an adapter P₁. The sequences comprise, at their 5′ end, an adapter P₂. The adapter P₁ forms part of the capture species 26 grafted, by covalent bonding, onto the functionalization surface. The adapter P₂ of each sequence is free.

The reaction medium 2 is placed in a fluidic chamber 4, in contact with the interface layer 23. The sequences to be characterized and the reaction medium are isolated from the nanowires 30 by the interface layer 23. In this example, the configuration is that in which the reaction medium contains various types of bases labelled with fluorescent labels.

In each cycle, a light source 5 illuminates the functionalization surface 25, in an excitation spectral band centred on a fluorescence excitation wavelength of a fluorescent label.

Below, reference will be made to the sequence S₁ captured and amplified at capture site 25₁. In a first cycle, a base T was hybridized. FIG. 1E shows a second cycle, in which a base C labelled with a fluorescent label C*, represented by the symbol *, hybridizes to a base G of the sequence. The objective of the device is to detect the fluorescent label C*, by means of a transduction of the fluorescence light.

The light source 5 generates excitation light 7, which propagates through the sample 2 to the functionalization surface 25. Under the effect of the illumination at the excitation wavelength, fluorescence light 8 is emitted by the fluorescent label C*, at a fluorescence wavelength longer than the excitation wavelength. Fluorescence photons are emitted, forming fluorescence light 8. Because of the transparency of the interface layer 23, or of the material 22 from which the electrode 21_{c} is made, certain fluorescence photons propagate through a nanowire 30. When a fluorescence photon is absorbed at the junction 33, electron/hole pairs are formed. Under the effect of the potential difference between the first portion 31 and the second portion 32, the electrons propagate through the n-doped portion, toward the higher potential while the holes propagate in the opposite direction through the p-doped portion. This results in an increase in the electric current flowing through the detection circuit 40.

The choice of the semiconductor forming the nanowire 30 depends on the absorption spectral band of said material, said absorption spectral band needing not only to contain the fluorescence wavelength, but also preferably not to contain the excitation wavelength of the fluorescent label. The nanowire thus acts as a nanophotodetector of the fluorescence light, while not being sensitive to the excitation wavelength. Each nanowire thus forms a filter with respect to the excitation wavelength. Optionally, an absorbent layer, for example a zinc oxide layer deposited by sputtering, may be added between the functionalization surface 25 and the conductive layer 22, to reduce the spectral width of the response of the nanowires. When the semiconductor is GaAs, the fluorescent label may be Cy3 (cyanine): excitation wavelength 540 nm and emission wavelength between 555 and 600 nm.

Preferably, the diameter of the nanowires is set so as ensure sensitivity is restricted to a narrow spectral band. By narrow spectral band, what is meant is a bandwidth typically of a few tens of nm, and preferably less than 100 nm. The bandwidth corresponds to the full width at half maximum of the absorption peak in the absorption spectrum. The detection bandwidth is for example of the order of 50 nm. It is thus possible to adjust the detection spectral band so that it contains the fluorescence wavelength of the fluorescent label and so that it does not contain the excitation wavelength of the fluorescent label. The ability of nanowires to form a wavelength-selective photodetector has been described in Mokkapati et al., “Optical design of nanowire absorbers for wavelength selective photodetectors”, Sci. Rep. 2015, 5 15339. Spectral detection sensitivity may be adjusted by incorporating quantum wells or quantum dots into the junction 33. This makes it possible to obtain a junction 33 the composition of which is different from the remainder of the nanowire. The detected wavelength then corresponds to the wavelength of the gap defined by the quantum well or quantum dot.

Each nanowire thus forms a nanophotodetector. In each cycle, it is necessary not only to detect fluorescence light, but also to identify the fluorescent label that generated the fluorescence light, in order to identify the base to which it was bound. In this example, it is assumed that the nucleic bases contained in the reaction medium are labelled with a fluorescent label A*, G* and C*, respectively. It is assumed that the T bases are not labelled with a fluorescent label. In each cycle, the following is detected:
either the absence of fluorescence light, resulting in an absence of variation in the current delivered by the nanowires: this corresponds to hybridization of a T base;
or fluorescence light, the labels A*, G* and C* being discriminated between.

One important aspect of the invention is that discrimination is achieved by characterizing the detection electric current delivered by the nanowires.

In order to discriminate between the fluorescence light respectively emitted by each fluorescent label, each fluorescent label used has one or more emission parameters that are different from those of the others. By emission parameter, what is meant is a parameter characterizing a temporal distribution of the fluorescence intensity emitted by the fluorescent label. It may for example be a question of the maximum intensity Iₘₐₓ of the distribution and/or the decay time of the fluorescence light. The decay time of the fluorescence light corresponds to a time for which the fluorescence intensity decreases between two predetermined levels, for example between the maximum intensity Iₘₐₓ and a certain percentage of the maximum intensity, for example 50% or 80%. The decrease in fluorescence intensity may vary as

$${e}^{\frac{- t}{τ}} ,$$

where t corresponds to time and τ is a decay constant. In this case, the decay time may be characterized by the decay constant τ.

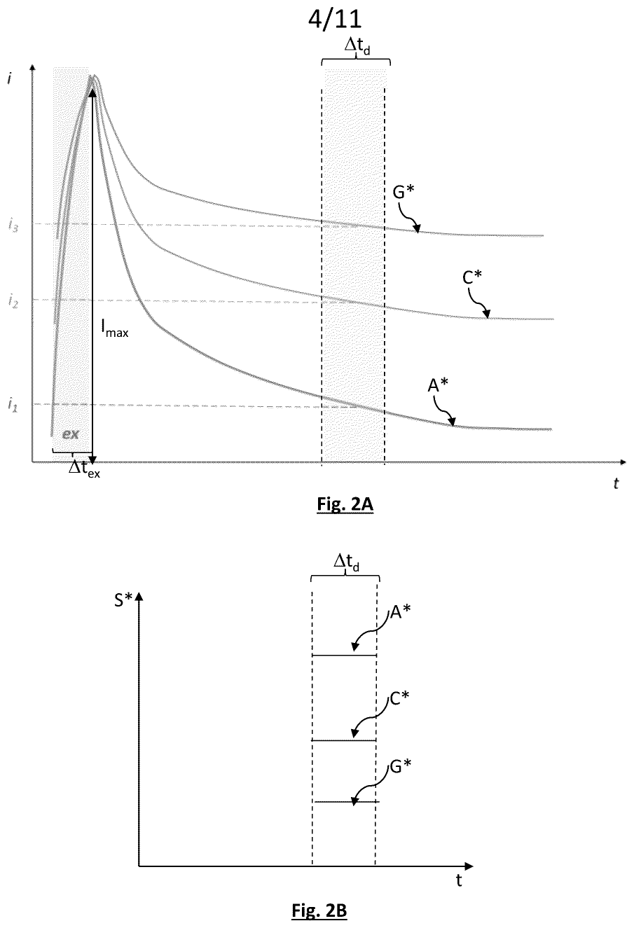

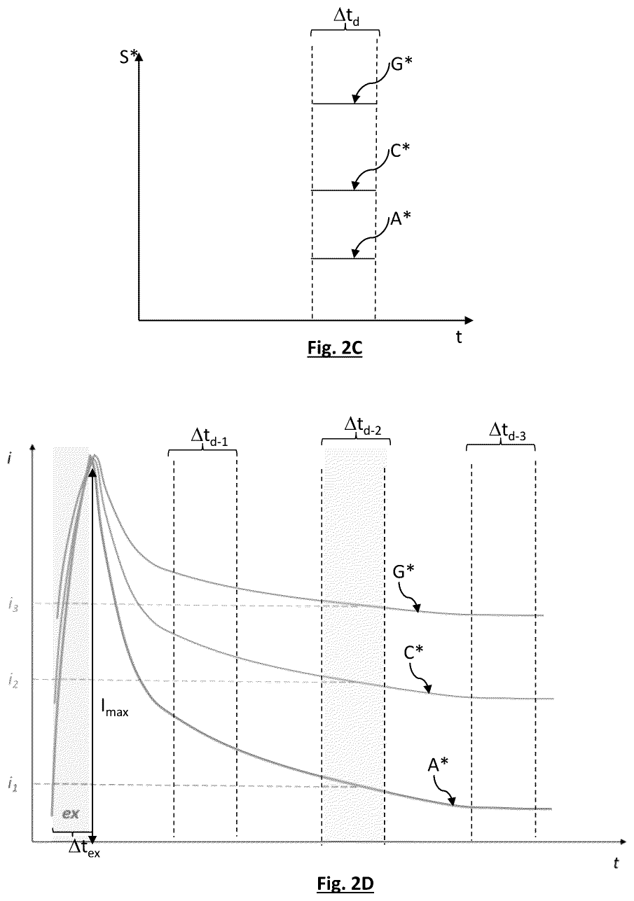

FIG. 2A schematically shows three temporal distributions of the fluorescence intensity of the three fluorescent labels G*, C* and A* labelling the bases G, C and A, respectively. In this example, the maximum intensity Iₘₐₓ is considered to be identical for all three fluorescent labels. However, the decay of A* is faster than the decay of C*, and the decay of C* is faster than the decay of G*. Thus, τ(A*)>τ(C*)>τ(G*). τ(A*) designates the decay constant of A*, and similarly τ(C*) and τ(G*). designate the decay constants of C* and G*, respectively.

The symbol S designates a detection signal delivered by the detection circuit 40 and formed from charge collected in each nanowire. The device comprises a processing unit 41, allowing the detection signal to be characterized, so as to identify the fluorescent label that generated the fluorescence light. The processing unit 41 may comprise a microprocessor 41, connected to a memory 42 in which instructions are stored. The processing unit forms a characterization signal S* from the detection signal.

FIG. 2A shows:
an excitation time period Δtₑₓ, which corresponds to the light pulse emitted by the light source 5. The excitation time period Δtₑₓ may for example be a few tens of ps or of the order of one ns.
a detection time period Δt_{d}, during which the detection signal S is characterized, to identify the fluorescent label having generated fluorescence light, or the absence of a fluorescent label. The detection time period Δt_{d} may for example be from 100 ps to 1 ns or a few ns. The detection period preferably occupies part of the decay of the fluorescence light emitted by the fluorescent label.

The emission of fluorescence light allows an exploitable detection signal to be obtained in a fluorescence time of the order of one or more ns.

FIG. 2B illustrates a first example of embodiment, in which the characterization signal corresponds to an absolute value of the time derivative of the amplitude of the electric current delivered by each nanowire. Thus,

$$S ( t ) = \vert \frac{di ( t )}{dt} \vert .$$

It may be seen that:
the characterization signal is maximum when the fluorescent label causing the fluorescence is A*;
the characterization signal is minimum when the fluorescent label causing the fluorescence is G*;
the characterization signal is between the maximum value and the minimum value when the fluorescent label causing the fluorescence is C*.

An absence of variation in the detection signal makes it possible to conclude that no fluorescence has been detected. This reflects hybridization of a T base to the sequence to be characterized.

The embodiment described with reference to FIG. 2B, which is based on a differential measurement of the fluorescence intensity, has the advantage of being insensitive to variability in the number of sequences respectively amplified at each capture site.

In a second example, shown in FIG. 2C, the processing unit is programmed to determine a characterization signal S* corresponding to an integral of the detection signal. S*=∫_{Δtd}S(t)dt. It may be seen that:
the characterization signal is maximum when the fluorescent label causing the fluorescence is G*;
the characterization signal is minimum when the fluorescent label causing the fluorescence is A*;
the characterization signal is between the maximum value and the minimum value when the fluorescent label causing the fluorescence is C*.

When the characterization signal corresponds to an integration of the detection signal, the level reached by the characterization signal allows the fluorescent labels G*, A* and C* to be discriminated between. The same goes when the characterization signal is a mean or a median or results from another statistical indicator representative of the intensity of the detection signal during the detection period.

However, the number of sequences to be characterized immobilized on the functionalization surface, plumb with a nanowire or with a given capture site, may vary, as may the number of amplification errors. Thus, the number of identical sequences replicated per amplification at each capture site may vary from one capture site to another capture site. Now, the amplitude of the photoinduced current depends on the number of fluorescent labels labelling bases hybridized in a given cycle. To address this difficulty, a calibration sequence Sc may be grafted onto each sequence to be characterized, so as to establish the intensity levels respectively associated with each fluorescent label.

The calibration sequence Sc corresponds to a succession of bases arranged in a known order, and the location of which in the adapter is known. For example, the calibration sequence is placed in a known location in the adapter P₂, which is intended to remain free after amplification. Use of a calibration sequence is particularly useful when the characterization signal corresponds to the detection signal at a given time, or comprises an integral of the detection signal.

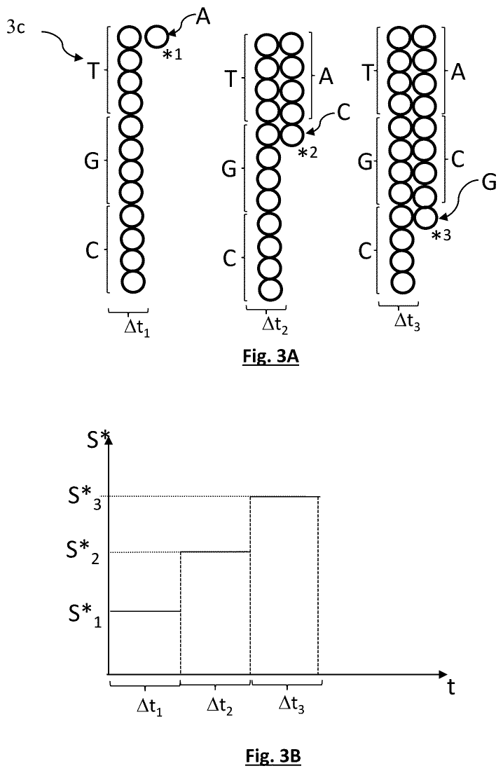

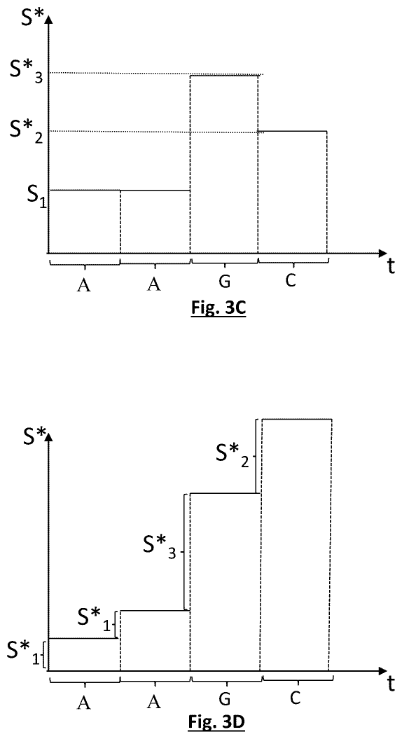

FIG. 3A illustrates one example of calibration sequences, composed of a succession of 4 T bases, 4 G bases and 4 C bases. In this example, the calibration sequence forms part of the adapter P₂. The characterization signal is an integral of the detection signal during a predetermined time period. During sequencing of the calibration sequence:
in a first period Δt₁, which corresponds to a period during which complementary A bases are successively hybridized to T bases of the calibration sequence, the intensity of the characterization signal corresponds to a first level S*₁. The fluorescent label A* has been symbolized by *1 in FIG. 3A.
in a second period Δt₂, which corresponds to a period during which complementary C bases are successively hybridized to G bases of the calibration sequence, the intensity of the characterization signal corresponds to a second level S*₂. The fluorescent label C* has been symbolized by *2 in FIG. 3A.
in a third period Δt₃, which corresponds to a period during which complementary G bases are successively hybridized to C bases of the calibration sequence, the intensity of the characterization signal corresponds to a third level S*₃. The fluorescent label G* has been symbolized by *3 in FIG. 3A.

FIG. 3B shows the change in the characterization signal in the periods Δt₁, Δt₂ and Δt₃. FIG. 3B corresponds to a configuration in which each fluorescent label comprises a terminator. During the period Δt₁, the signal S*₁ is measured in four successive cycles. During the period Δt₂, the signal S*₂ is measured in four successive cycles. During the period Δt₃, the signal S*₃ is measured in four successive cycles.

Use of the calibration sequence makes it possible to experimentally determine the levels S*₁, S*₂ and S*₃ at each capture site, knowing that the number of identical sequences may vary from one site to another. The levels thus established are used to interpret the detection signals detected in the cycles performed on each sequence to be characterized.

According to one variant, illustrated in FIG. 2D, the detection signal is characterized during a plurality of successive detection periods Δt_{d-1}, Δt_{d-2} and Δt_{d-3}. In such an embodiment, use of a calibration sequence is also preferable.

In each cycle, the processing unit 41 establishes a characterization signal S*, which characterizes the detection signal S measured across the terminals of the detection circuit 40, the detection signal resulting from an electric current formed in at least one nanowire. The characterization signal S* may comprise an integration or a derivative of the intensity measured by each nanowire taken into account. The processing unit compares the characterization to stored values or values established during the calibration period, each value being assigned to one fluorescent label.

The characterization signal makes it possible to identify, in the or each detection period, one fluorescent label among a plurality of fluorescent labels that are referred to as candidates (in this example A*, C* and G*), the characteristics of which, in terms of emission intensity or decay, are known. The characteristics may be stored in the memory 42. This allows the nucleic base hybridized in the cycle to be identified.

When three labelled bases are used, the following fluorescent labels are suited to implementation of the invention: DAPI (half-life 1.88 ns), Hoechst 33342 (half-life 1.05 ns) and Hoeschst 33258 (half-life 1.22 ns). The wavelengths at which excitation is maximum are 358 nm, 350 nm and 349 nm, respectively.

The embodiment in which the various types of complementary bases are employed simultaneously is particularly suited to implementation of the invention. The fluorescent labels bound to each base comprise a terminator, the latter being removed by cleaving following formation of the characterization signal. However, the invention may be implemented by exposing the sequences to be characterized to known bases in successive baths.

It will be noted that the fluorescent labels may be cleaved in each cycle, following formation of the characterization signal. However, this is not crucial.

FIG. 3C schematically shows the characterization signal resulting from sequencing of a TTCG sequence. In this example, the characterization signal corresponds to an integral of the detection signal during a predetermined detection period. Each base may be identified with reference to the signals S*₁, S*₂ and S*₃ established during the calibration. In this example, it has been assumed that, following each detection, the fluorescent label undergoes cleavage. The x-axis designates the detected base. The y-axis corresponds to the characterization signal.

FIG. 3D schematically shows the characterization signal resulting from the same sequence. In this example, the characterization signal corresponds to an integral of the detection signal during a predetermined detection period. Each base may be identified with reference to the signals S*₁, S*₂ and S*₃ established during the calibration and stored in the memory 42. In this example, no fluorescent-label cleavage is performed. The intensity of the fluorescence signal gradually increases, under the effect of accumulation of the fluorescent labels bound to bases hybridized to the sequence to be characterized.

It may also be useful to employ a calibration sequence when the characterization signal is a differential signal, of the time-derivative type. This may make it possible to calibrate the detection signal for the various bases. When the characterization signal is of the time-derivative type, it is preferable for the fluorescent labels to be cleaved following detection of hybridization.

Generally, use of the calibration sequence allows a response of the device at each capture site to be established. This makes it possible to take into account the fluorescence emission characteristics of each fluorescent label, the variability in the number of sequences amplified at each capture site, but also potential variability in the detection performance of each nanowire.

Preferably, the fluorescent labels are excited in the same excitation spectral band, this allowing the same light source to be used. Alternatively, the fluorescent labels are excited in different excitation spectral bands, this potentially requiring various light sources to be used.

Use of nanowires is particularly advantageous. It makes it possible to obtain an exploitable signal using a small number of sequences replicated at the same capture site. It makes it possible to reduce the size of the capture sites, relative to the prior art. It is thus possible to characterize, on a functionalization surface of given area, a larger number of sequences. The cycles may be implemented in parallel at each capture site defined on the functionalization surface. The reduction in the number of amplifications makes it possible to limit the drawbacks associated with the sequence amplification process: smaller volume of reagents required, smaller number of replicates and hence a smaller number of amplification errors.

Specifically, the detection circuit allows very low currents to be measured, of the order of a few tens of pA to a few nA. In addition, the nanowires may be closely spaced, typically with a spatial pitch of less than 500 nm (compared to 1.1-1.7 μm typically for a CMOS optical detector). This allows a device having a high ratio of working area (i.e. the area of the functionalization surface) to fluid volume to be obtained.

The excitation spectral band is preferably between 350 nm and 550 nm. The detection spectral band of each nanowire is preferably dimensioned to allow the fluorescence light to be detected while masking the excitation light. Alternatively, the detection currents, respectively formed in each nanowire during excitation and emission of fluorescence light, may be temporally separate. Advantage is taken of the fast response time (of the order of one ps) of the nanowires, as described in the publication Gallo, et al., “Picosecond response times in GaAs/AlGaAs core/shell nanowire-based photodetectors” Appl. Phys. Lett. 2011, 98 (24) 241113.

Various aspects of the design of the device according to the invention will now be described.

### Nanowire Formation

FIGS. 4A to 4E schematically show steps of forming nanowires from a silicon substrate 10. FIGS. 4A to 4E correspond to a so-called bottom-up approach. The substrate 10 is formed from Si, of (111) orientation and comprises a surface layer 101 of SiO₂, which is intended to form a barrier layer, of 10 nm-15 nm thickness. The SiO₂ layer is covered with an electrosensitive PMMA layer 102 of 45-80 nm thickness. The layers 101 and 102 are patterned by photolithography and etched, respectively, so as to form nanowells that are isolated from one another, in a predetermined pattern. See FIG. 4A. The nanowells open onto the Si substrate. A thin metal layer 103, for example of gold, of 5 to 10 nm thickness is deposited on the SiO₂ layer. The metal added, in this case gold, acts as a catalyst. See FIG. 4B. The excess gold between the nanowells is removed by lift-off of the PMMA layer 102. See FIG. 4C. Gold islands 103 that are isolated from one another are thus obtained, in positions corresponding to the positions of the nanowells formed beforehand.

After heating to a temperature greater than 450° C., the islands form droplets. The gold droplets act as catalysts. The semiconductor nanowires are then formed by molecular beam epitaxy (MBE). This consists in sending one or more molecular beams toward the substrate, to achieve epitaxial growth. See FIG. 4D. The molecular vapor beams contain the chemical species from which the semiconductor nanowires are made (for example Ga and As to form GaAs) and the dopants. The atomic species adsorb to and diffuse across the surface of the Si substrate in the form of adatoms. The adatoms are incorporated into the gold droplets. When the latter saturate, nucleation of the nanowires occurs first at the interface between the droplet and substrate, then at the interface between the droplet and the growing nanowire.

The process is not limited to use of gold as catalyst. Other catalysts may be used, for example Ga or Sn. Self-catalyzed nanowire growth is referred to when the catalyst is Ga and Ga is also a constituent of the semiconductor.

This process for example allows nanowires to be grown using As, Ga, and C and Si as p- and n-dopant, respectively, the growth temperature being 600° C.-610° C. The process continues until the nanowires reach a predetermined height. See FIG. 4E.

Following the step shown in FIG. 4E, the encapsulation layer 15 is formed between the nanowires, for example by spin coating. The encapsulation layer allows the nanowires to be electrically insulated from one another, and increases the mechanical strength of the assembly. Chemical etching, plasma etching and/or polishing may be carried out on the end of the nanowires opposite the substrate, so as to remove residues of the catalyst and increase the uniformity of the height of the nanowires and of the encapsulation layer 15.

The conductive layer 22, then the interface layer 23, are then successively deposited on the assembly formed by the nanowires and the encapsulation layer 15. In the embodiment illustrated in FIGS. 4A to 4E, formation of the nanowells on the substrate 10 allows the position of the nanowires to be controlled. Thus, the nanowires may be arranged regularly, for example in patterns of square or hexagonal unit cell. The pitch between two adjacent nanowires may be small, of the order of twice the diameter, or may be larger, for example a few to several hundred nm.

Another advantage of the bottom-up approach described with reference to FIGS. 4A to 4E is that it allows more precise control of the crystal structure of the nanowires. The bottom-up approach allows controlled incorporation of quantum dots or quantum wells, while controlling their position and composition, in particular along the axis Z.

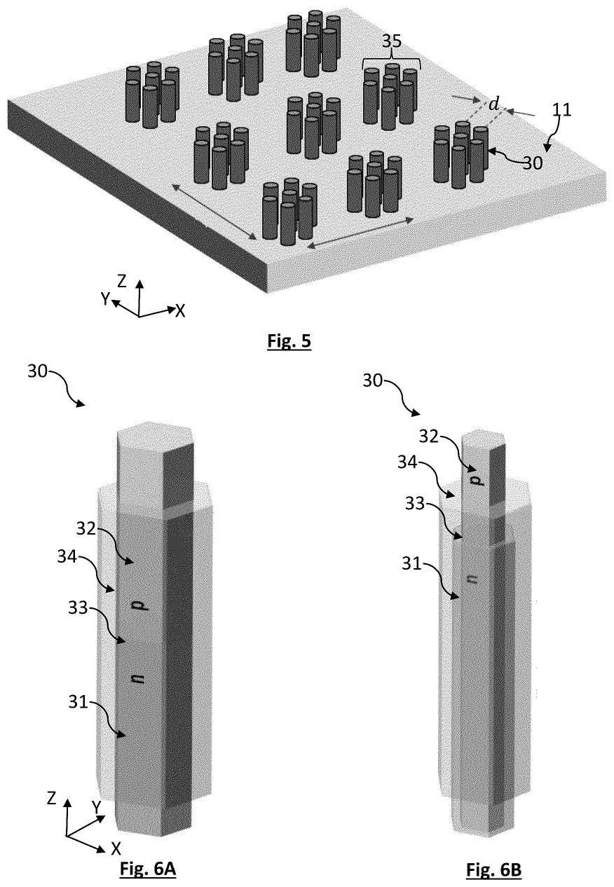

FIG. 5 illustrates a configuration in which the nanowires are arranged to form clusters 35. The nanowires of a given cluster are closely spaced, the distance d between two adjacent nanowires of a given cluster preferably being larger than or equal to the diameter of the nanowires. The distance between two adjacent clusters may be equal to or larger than twice the distance d.

When the nanowires are distributed in clusters as described with reference to FIG. 5, the nanowires of a given cluster are preferably connected to the same electrode, both on the substrate 10 and on the multilayer structure 20. The nanowires of a given cluster address the same capture site. The nanowires of a given cluster are thus functional simultaneously. Nanowires not connected to the detection circuit are not functional. Grouping the nanowires into clusters allows advantage to be taken of the potential to detect, with a plurality of adjacent nanowires, fluorescence resulting from hybridization of bases of the same type to sequences of the same type.

Because of the high sensitivity of each nanowire, the number of nanowires composing a given cluster may be relatively small. Thus, the area occupied, in the plane P_{XY}, by each cluster is small, each cluster selectively addressing a nucleotide sequence different from the other clusters. It is thus possible to provide a large number of clusters, respectively addressing various nucleotide sequences, in the same compact device.

Arrangement of the nanowires into clusters 35 may be combined with structuring the functionalization surface to form nanowells 27, as described with reference to FIG. 1C. In this case, each nanowell 27 lies facing the nanowires belonging to a given cluster 35.

According to another possibility, the nanowires are obtained by etching, using a so-called top-down approach. The top-down approach is described in patent application FR2114563 filed on Dec. 27, 2021.

### Structure of the Nanowires

FIGS. 6A and 6B show other nanowire structures able to be implemented in a device according to the invention. FIG. 6A shows a nanowire similar to the nanowires described above. The junction 33 is placed axially between two portions 31, 32 of different doping, and which are spaced apart from each other along the transverse axis Z. In the example of FIG. 6A, a passivation cladding 34 encircles the nanowire.

In the example of FIG. 6B, the junction 33 extends radially between two zones of different doping. Thus, the junction 33 extends around the transverse axis Z, parallel to the latter. The first portion 31 and the second portion 32 are separated radially, the separation between the two portions corresponding to a separation radius. The first portion extends between the axis of the nanowire and the junction 33, whereas the second portion extends around the junction 33.

An axial structure is considered to be advantageous because it favours incorporation of quantum wells or quantum dots into the nanowires, in order to adjust the absorption spectrum.

A radial structure makes it possible to provide a junction 33 extending a considerable height along the axis Z, this allowing detection sensitivity to be increased. Optionally, the radial structure shown in FIG. 6B comprises an annular cladding 34 such as described with reference to FIG. 6A.

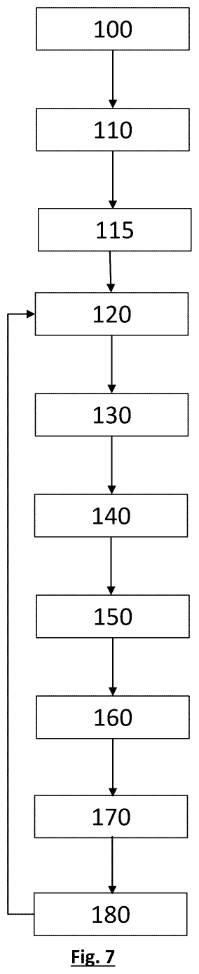

FIG. 7 schematically shows the main steps of a method for implementing the invention.

Step 100: providing a sample containing nucleic acid sequences drawn from a library prepared beforehand from the DNA strand to be sequenced, which is brought into contact with the functionalization surface 25.

Step 110: capturing nucleic acid sequences by means of the functionalization surface, preferably at capture sites arranged plumb with the nanowires.

Step 115: amplifying, for example bridge amplifying, the nucleic acid sequence captured at each site. Step 115 is optional, but it allows, in each cycle, a detection signal of higher intensity to be obtained, because of the increase in fluorescence light.

Step 120: adding a reaction medium, containing nucleic bases labelled with fluorescent labels and a hybridase. Preferably, a single nucleic base is capable of being hybridized to each sequence. Each type of base is labelled with a fluorescent label called the predetermined candidate fluorescent label, which is coupled to a terminator. Bases of the same type are labelled with the same candidate fluorescent label.

Step 130: rinsing the sample.

Step 140: illuminating the functionalization surface, so as to cause fluorescence of fluorescent labels hybridized following step 120.

Step 150: detecting a detection signal across the terminals of the detection circuit.

Step 160: characterizing the detection signal, so as to identify the fluorescent label among the candidate fluorescent labels.

Step 170: cleaving the terminator, optionally cleaving the fluorescent label, and rinsing the sample.

Step 180: reiterating steps 120 to 170, until each nucleotide sequence captured at a capture site has been sequenced.

In the method described with reference to FIG. 7, in step 120, the reaction medium contains a mixture of bases. As indicated above, in each cycle, 4 successive baths may be provided, respectively comprising the 4 types of bases labelled with fluorescent labels. The latter may then be identical or different from one another.

The device takes advantage of a fast response time, typically of the order of one ns.

It will be noted that the device does not require bulky optical components to be used. In addition, the response of the device is stable, and insensitive to environmental variations: pH of the sample, temperature, presence of molecules or ions other than the biomolecule of interest. This is due to the fact that the nanowires do not make contact with the sample, but are physically and electrically isolated from the latter by the interface layer 23.

Lastly, since the device is based on nanophotodetectors, it allows a compact analysis platform to be obtained. A wafer-level manufacturing process may be used to obtain the device, this allowing cost to be lowered.

1. A device for identifying a fluorescent label, the fluorescent label being configured to emit fluorescence light in a fluorescence spectral band, the device comprising:
a substrate, comprising at least one first electrode;
a multilayer structure, comprising at least one second electrode;
nanowires, extending between the first electrode and the second electrode, parallel to a transverse axis;
an encapsulation layer extending around the nanowires, between the substrate and the multilayer structure, the encapsulation layer being formed from an insulating material;
the multilayer structure comprising:
a conductive layer, forming each second electrode;
an electrically insulating interface layer covering each second electrode, each second electrode being interposed between the interface layer and one nanowire, the interface layer being bounded by a functionalization surface, the interface layer being configured to be placed between a sample, comprising the fluorescent label, and the second electrode, such that the functionalization surface forms an interface between the device and the sample;
the multilayer structure being such that the second electrode and the interface layer are transparent in a detection spectral band containing the fluorescence spectral band;
wherein:
each nanowire comprises a homojunction, or a heterojunction, or a Schottky junction between the first electrode and the second electrode;
the first electrode and the second electrode are configured to be connected to a detection circuit;
in such a way that each nanowire forms a nanophotodetector of the fluorescence light when the fluorescent label is bound to the functionalization surface, the light detected by each nanowire inducing an electrical detection signal in the detection circuit;
the device comprising a processing unit, programmed to:
acquire the detection signal during a detection time period;
determine a characteristic of the detection signal during the detection time period;
identify the fluorescent label depending on the characteristic.

2. The device according to claim 1, comprising a light source configured to emit excitation light in the excitation spectral band of the fluorescent label.

3. The device according to claim 1, wherein:
the light source is configured to emit excitation light during an excitation time period;
the detection time period is subsequent to the excitation time period.

4. The device according to claim 3, wherein:
following the excitation time period, the intensity of the fluorescence light grows then decays;
the characteristic of the detection signal is representative of the decay of the intensity of fluorescence light.

5. The device according to claim 1, wherein the functionalization surface is configured to capture a strand forming a chain of oligonucleotides.

6. The device according to claim 1, wherein the functionalization surface is segmented into various capture sites, each capture site being configured to capture one strand forming a chain of oligonucleotides.

7. The device according to claim 6, wherein:
the interface layer comprises, stacked one on top of the other, two sub-layers forming a lower sub-layer and an upper sub-layer, the lower sub-layer being interposed between the conductive layer and the upper sub-layer;
the upper sub-layer comprises wells that open into the lower sub-layer, each well being placed facing one nanowire, each well forming part of the functionalization surface;
the functionalization surface is segmented at the level of each well, so that each well forms one capture site.

8. The device according to claim 1, comprising a plurality of nanowires, extending between the same first electrode and the same second electrode, the nanowires forming a nanowire cluster.

9. The device according to claim 1, comprising a plurality of nanowire clusters spaced apart from one another, such that one nanowire of a cluster is closer to another nanowire of said cluster than to another nanowire of another cluster, the nanowires of a given cluster extending between the same first electrode and the same second electrode.

10. The device according to claim 1, comprising a plurality of nanowires, wherein:
a plurality of first electrodes are formed on the substrate, and a plurality of second electrodes are formed on the multilayer structure, each nanowire extending between a first electrode and a second electrode;
each first electrode is connected to a first addressing unit, configured to select at least one first electrode;
each second electrode is connected to a second addressing unit, configured to select at least one second electrode;
so that the detection circuit detects a detection current induced by each nanowire extending between the selected first electrode and the selected second electrode.

11. A method for identifying a fluorescence label using a device according to claim 1, the fluorescent label being capable of emitting fluorescence light, in the detection spectral band, when it is illuminated by excitation light, the functionalization surface being configured to capture a strand of nucleic acid, the method comprising:
a) placing a sample, comprising nucleic acids, in contact with the functionalization surface;
b) capturing at least one strand of nucleic acid on the functionalization surface;
c) adding nucleic bases to the sample, at least two different nucleic bases being labelled with two different fluorescent labels respectively;
wherein the sample comprises active principles configured to allow hybridization of a nucleic base to the strand of nucleic acid captured on the functionalization surface,
wherein the method further comprises:
d) exposing the functionalization surface to excitation light, in an excitation spectral band of at least one fluorescent label;
e) following step d), detecting a detection signal across terminals of the detection circuit, during a detection time period;
f) depending on the detection signal detected in step e), identifying the fluorescent label;
g) reiterating steps c) to f) so as to gradually hybridize nucleic bases along the strand of nucleic acid.

12. The method of claim 11, wherein step b) comprises amplifying each captured strand of nucleic acid.

13. The method of claim 11, wherein steps b) to g) are carried out at various capture sites distributed over the functionalization surface.

14. The method of claim 11, wherein step f) comprises:
determining a characteristic of the detection signal during the detection time period;
identifying the fluorescent label depending on the characteristic.

15. The method of claim 14, wherein:
the fluorescent label is chosen from a plurality of candidate fluorescent labels;
step f) comprises selecting the fluorescent label from the candidate fluorescent labels depending on the characteristic of the detection signal.

16. The method of claim 11, wherein step f) comprises estimating a time derivative of the detection signal.

17. The method of claim 11, wherein step f) comprises detecting an intensity level or integrating the detection signal during at least one predetermined time period.

18. The method of claim 11, wherein:
prior to step a), each nucleotide sequence is bound to a known calibration sequence;
steps c) to f) are implemented so as to hybridize the bases of the calibration sequence, steps c) to f) forming a calibration phase;
the detection signal obtained in each step e) of the calibration phase is used to calibrate a response of the device to the bases of the calibration sequence.

19. The method of claim 11, wherein:
each candidate fluorescent label emits fluorescence light with a fluorescence intensity that grows then decays;
the decay of the fluorescence intensity of each fluorescent label is characterized by a decay constant;
the decay constants of two different fluorescent labels are different.

20. The method of claim 11, comprising, following step g), a step h) of identifying the hybridized nucleic acid base.

21. The method of claim 20, wherein
following step h), the fluorescent label is cleaved and the sample is rinsed;
after rinsing, steps c) and h) are reiterated, so as to identify a nucleotide sequence forming the captured strand of nucleic acid.
